(19) United States
(12) Patent Application Publication
(10) Pub. No.: US 2026/0335267 A1
(43) Pub. Date: Oct. 29, 2026

(54) COMPOSITIONS AND METHODS FOR NASAL BIOFILM DISRUPTION AND PATHOGEN REDUCTION

(71) Applicant: Robb Finnemore, Ponte Vedra Beach, FL (US)

(72) Inventor: Robb Finnemore, Ponte Vedra Beach, FL (US)

(21) Appl. No.: 19/651,915

(22) Filed: Apr. 20, 2026

Related U.S. Application Data
(60) Provisional application No. 63/793,590, filed on Apr. 23, 2025.

(51) Int. Cl. A61K 31/198; A61K 9/00; A61K 31/047; A61K 33/18; A61K 33/38; A61K 36/752; A61P 31/04

(52) U.S. Cl. CPC A61K 31/198; A61K 9/0043; A61K 31/047; A61K 33/18; A61K 33/38; A61K 36/752; A61P 31/04

(57) ABSTRACT

A nasal or intranasally administrable composition and related methods for disrupting microbial biofilms and reducing pathogenic colonization in the nasal cavity and/or sinonasal passages are provided. The composition comprises ethylenediaminetetraacetic acid (EDTA) or a salt thereof, xylitol, and one or more antimicrobial agents in an aqueous carrier. In certain embodiments, EDTA is present from about 0.05% to about 2.0% by weight and xylitol is present from about 1% to about 20% by weight, and the antimicrobial agent comprises colloidal silver, grapefruit seed extract, and/or povidone iodine. In certain aspects, a two-step approach is provided in which a first intranasal administration comprising EDTA and xylitol weakens biofilms and a second intranasal administration comprising an antimicrobial agent reduces or eliminates exposed pathogens.

### CROSS-REFERENCE TO RELATED APPLICATION

This application claims priority to U.S. Provisional Application Ser. No. 63/793,590 filed Apr. 23, 2025, entitled “COMPOSITIONS AND METHODS FOR NASAL BIOFILM DISRUPTION AND PATHOGEN REDUCTION, which is hereby incorporated in its entirety.

### FIELD

The present invention relates to nasal and sinonasal compositions formulated for disrupting microbial biofilms and reducing pathogenic colonization in the nasal cavity and/or sinonasal passages. More particularly, the invention relates to aqueous intranasal formulations comprising a combination of ethylenediaminetetraacetic acid (EDTA), xylitol, and antimicrobial agents for the treatment of biofilm-associated nasal and sinus conditions.

### BACKGROUND

Chronic rhinosinusitis (CRS) affects millions of individuals worldwide and represents a significant healthcare burden. Recent research has identified bacterial biofilms as a major contributing factor in the pathogenesis and persistence of chronic sinus infections and inflammatory conditions. Biofilms are complex communities of microorganisms encased in a self-produced extracellular polymeric matrix that adheres to biological surfaces, including the nasal and sinus mucosa.

The biofilm mode of growth provides bacteria with significant survival advantages, including increased resistance to antimicrobial agents and host immune defenses. Studies have shown that bacteria within biofilms can be 10 to 1,000 times more resistant to antibiotics compared to their planktonic counterparts. This enhanced resistance is attributed to multiple factors, including reduced penetration of antimicrobials through the biofilm matrix, altered microenvironments within the biofilm, and the presence of persister cells.

In the context of chronic rhinosinusitis, biofilm formation has been particularly associated with Staphylococcus aureus, including methicillin-resistant S. aureus (MRSA), as well as other organisms such as Pseudomonas aeruginosa.A specific concern in patients with Chronic Inflammatory Response Syndrome (CIRS) and mold-related illness is the presence of Multiple Antibiotic Resistant Coagulase Negative Staphylococci (MARCoNS) in the nasal passages. MARCoNS form particularly resilient biofilms that are difficult to eradicate with conventional therapies.

Existing antimicrobial nasal sprays and irrigation solutions often fail to adequately penetrate or dismantle the biofilm matrix, allowing pathogens to persist despite treatment. Traditional saline rinses may provide temporary symptomatic relief but do not address the underlying biofilm problem. Systemic antibiotics, even when appropriately selected based on culture and sensitivity testing, frequently fail to achieve therapeutic concentrations within biofilms and may contribute to the development of antimicrobial resistance.

Several approaches have been investigated for biofilm disruption, including the use of chelating agents, surfactants, enzymes, and antimicrobial peptides. Ethylenediaminetetraacetic acid (EDTA), a chelating agent that binds divalent cations such as calcium and magnesium, has shown promise in disrupting biofilm architecture by destabilizing the extracellular matrix. However, EDTA alone is often insufficient to completely eradicate established biofilms.

Xylitol, a five-carbon sugar alcohol, has demonstrated antibacterial and anti-biofilm properties in various studies. The mechanism of action appears to involve interference with bacterial adhesion and biofilm formation, as well as potential osmotic effects. Xylitol has been shown to be particularly effective against S. pneumoniae and has been used in nasal sprays for the prevention of acute otitis media.

Colloidal silver, consisting of silver nanoparticles suspended in solution, has a long history of use as an antimicrobial agent. Recent studies have demonstrated significant anti-biofilm activity of colloidal silver against S. aureus biofilms in vitro and in animal models. The antimicrobial mechanism involves multiple targets, including disruption of bacterial cell membranes, interference with DNA replication, and generation of reactive oxygen species.

Grapefruit seed extract (GSE) contains various bioactive compounds, including flavonoids and phenolic compounds, with demonstrated antimicrobial activity against a broad spectrum of microorganisms. GSE has shown activity against both Gram-positive and Gram-negative bacteria, as well as fungi and some viruses.

Despite these individual advances, there remains a need for an effective nasal spray formulation that can both disrupt established biofilms and prevent their reformation while maintaining safety for regular use in the nasal passages. The ideal formulation would combine multiple mechanisms of action to achieve synergistic effects against biofilm-associated infections.

### SUMMARY

The present invention provides intranasally administrable compositions and methods for disrupting microbial biofilms and reducing pathogenic colonization in the nasal cavity and/or sinonasal passages. The compositions combine multiple active agents that work synergistically to degrade biofilm structure, eliminate pathogens, and support restoration of healthy nasal microbiome.

In one aspect, the invention provides an intranasally administrable composition comprising: (i) ethylenediaminetetraacetic acid (EDTA) or a pharmaceutically acceptable salt thereof; (ii) xylitol; (iii) at least one antimicrobial agent; and (iv) an aqueous carrier.

In certain embodiments, EDTA is present in an amount from about 0.05% to about 2.0% by weight, and xylitol is present in an amount from about 1% to about 20% by weight.

In certain embodiments, the antimicrobial agent comprises colloidal silver in an amount from about 1 ppm to about 300 ppm, grapefruit seed extract (GSE) in an amount from about 0.05% to about 2.0% by weight, povidone iodine in an amount from about 0.01% to about 2.0% by weight, one or more essential oils or plant-derived phenolics, antimicrobial peptides, or combinations thereof.

In preferred embodiments, the composition includes: 0.5% to 1.0% by weight disodium EDTA; 7.5% to 10% by weight xylitol; 20 ppm to 130 ppm colloidal silver; 0.5% to 1% by weight grapefruit seed extract; and sterile water to 100%.

In certain embodiments, the composition further includes sodium chloride at a concentration sufficient to produce an isotonic solution, preferably about 0.9% by weight.

In other embodiments, the composition includes a buffering agent to maintain physiological pH. The buffering agent may include sodium bicarbonate, and the composition preferably has a pH between 6.0 and 7.0, more preferably between 6.3 and 6.8, and most preferably about 6.5.

In alternative embodiments, the invention provides a silver-free formulation including EDTA, xylitol, and grapefruit seed extract. Povidone iodine can be included as an alternative antimicrobial agent and sterile water to 100%.

In another aspect, the invention provides a two-step intranasal treatment system and method in which: Step 1 weakens and/or disrupts biofilms using EDTA and xylitol; and Step 2 eliminates exposed pathogens using antimicrobial agents such as colloidal silver, grapefruit seed extract, povidone iodine, and/or other antimicrobial agents (including natural antimicrobials). The first and second compositions may be administered sequentially, concurrently, or separated by a time interval ranging from minutes to hours, depending on treatment protocol.

The invention also provides methods for treating biofilm-associated nasal and sinonasal conditions by administering to a subject an effective amount of the composition(s) described herein, where dosing may be one or more actuations or volumes per nostril or per nasal cavity, and may be repeated according to patient need and tolerability.

### DETAILED DESCRIPTION OF THE INVENTION

The present invention provides improved intranasal compositions for the treatment of biofilm-associated nasal and sinonasal conditions. The compositions combine multiple active agents that work through complementary mechanisms to achieve superior biofilm disruption and pathogen elimination compared to single-agent approaches.

As used herein, the term “biofilm” refers to a community of microorganisms embedded in a self-produced extracellular polymeric matrix adhered to a biological or abiotic surface.

The term “EDTA” refers to ethylenediaminetetraacetic acid or pharmaceutically acceptable salts thereof, including but not limited to disodium EDTA, calcium disodium EDTA, and tetrasodium EDTA.

The term “colloidal silver” refers to a suspension of silver nanoparticles in an aqueous medium, typically with particle sizes ranging from 1 to 100 nanometers.

The term “MARCoNS” refers to Multiple Antibiotic Resistant Coagulase Negative Staphylococci, which are antibiotic-resistant bacteria commonly found in patients with Chronic Inflammatory Response Syndrome.

The term “effective amount” refers to an amount of the composition sufficient to produce a therapeutic response, such as reduction in biofilm burden, decreased bacterial colonization, or improvement in clinical symptoms.

The term “intranasal administration” and “nasal spray” broadly include mist, aerosol, pump-based delivery, drops, rinse, irrigation, lavage, and nebulized delivery to the nasal cavity and/or sinonasal passages.

The compositions described herein may be regulated as pharmaceutical, medical, or wellness products depending on jurisdiction, formulation, labeling, and intended use. While not wishing to be bound by theory, the observed anti-biofilm and antimicrobial effects are believed to arise from one or more of the mechanisms described below.

In various embodiments, the invention is practiced as a two-step biofilm management protocol. Without being bound by theory, a first intranasal administration comprising EDTA and xylitol can weaken the biofilm matrix and/or reduce adhesion, thereby improving susceptibility of resident microorganisms to a subsequent antimicrobial administration. The subsequent antimicrobial administration can include colloidal silver, grapefruit seed extract, povidone iodine, one or more essential oils, plant-derived phenolics, antimicrobial peptides, or other natural antimicrobial agents suitable for intranasal use.

EDTA functions as a chelating agent that binds divalent cations, particularly calcium and magnesium ions that are essential for biofilm structural integrity. By sequestering these ions, EDTA destabilizes the biofilm matrix, increasing permeability and allowing penetration of antimicrobial agents. EDTA also has direct antimicrobial activity against certain bacteria by disrupting the outer membrane of Gram-negative organisms.

In some embodiments, the composition contains about 0.05% to about 2.0% by weight of EDTA (e.g., disodium EDTA). In preferred embodiments, the composition contains 0.5% to 1.0% by weight of disodium EDTA. More preferably, the concentration is 0.5% to 0.75%, and most preferably about 0.5%. Higher concentrations may cause nasal irritation, while lower concentrations may be insufficient for biofilm disruption.

Xylitol is a five-carbon sugar alcohol that exhibits multiple beneficial properties in the nasal environment. It interferes with bacterial adhesion to epithelial cells, disrupts biofilm formation, and creates an osmotic effect that helps flush bacteria from the nasal passages. Xylitol also provides a moisturizing effect and has a cooling sensation that can improve patient comfort and compliance.

In some embodiments, the composition contains about 1% to about 20% by weight xylitol. In preferred embodiments, the composition contains 7.5% to 10% by weight xylitol. More preferably, the concentration is about 7.5% to 8.5%, which provides optimal anti-biofilm activity while maintaining isotonicity and avoiding excessive sweetness that could drain into the pharynx.

Colloidal silver provides broad-spectrum antimicrobial activity through multiple mechanisms, including disruption of bacterial cell membranes, interference with bacterial enzyme systems, and DNA damage. Silver nanoparticles have been shown to penetrate biofilms and maintain antimicrobial activity within the biofilm matrix. Unlike antibiotics, bacteria do not readily develop resistance to silver.

In embodiments containing silver, the composition contains about 1 ppm to about 300 ppm colloidal silver. In preferred embodiments, the composition contains 20 ppm to 130 ppm colloidal silver, more preferably 25 ppm to 50 ppm, and most preferably about 30 ppm. The silver nanoparticles preferably have a size range of 1 to 30 nanometers for optimal antimicrobial activity and stability.

Grapefruit seed extract (GSE) contains bioactive compounds including naringenin, limonoids, and other flavonoids with antimicrobial properties. GSE disrupts bacterial cell membranes and interferes with bacterial enzyme systems. It has shown activity against biofilm-forming bacteria and provides additional antimicrobial coverage.

In some embodiments, the composition contains about 0.05% to about 2.0% by weight grapefruit seed extract. In preferred embodiments, the composition contains 0.5% to 1.0% by weight grapefruit seed extract, more preferably about 0.5%. The extract is preferably standardized to contain consistent levels of active compounds.

The composition may include sodium chloride to achieve isotonicity with nasal secretions. However, xylitol may provide a cooling effect that may eliminate the need for sodium chloride to achieve patient comfort. When included, sodium chloride is preferably present at about 0.9% by weight.

The composition may include buffering agents to maintain physiological pH. Sodium bicarbonate is a preferred buffer, though some formulations may not require buffering due to the properties of xylitol. When included, the buffer maintains pH between 6.0 and 7.0.

The composition may include preservatives to maintain sterility during multi-use applications. Suitable preservatives include benzalkonium chloride, phenylethyl alcohol, or potassium sorbate. However, the antimicrobial agents in the formulation may provide sufficient preservation without additional preservatives. Also, in certain embodiments, the composition is preservative-free and is packaged in a sterile, multi-dose container relying on antimicrobial components to maintain microbiological stability.

In silver-free formulations, povidone iodine may be included as an alternative antimicrobial agent at concentrations of 0.01% to 2.0%.

### FORMULATION EXAMPLES

### Example 1: Colloidal Silver Formulation

Disodium EDTA: 0.5%
Xylitol: 7.5%
Colloidal silver (30 ppm): 0.003%
Grapefruit seed extract: 0.5%
Sterile water: to 100%
pH adjusted to 6.5 if needed

### Example 2: Silver-Free Formulation

Disodium EDTA: 0.5%
Xylitol: 7.5%
Grapefruit seed extract: 0.5%
Povidone iodine: 0.5%
Sterile water: to 100%
pH adjusted to 6.5 if needed

The intranasal compositions are prepared using standard pharmaceutical compounding techniques under aseptic conditions. The aqueous base is prepared by dissolving EDTA in sterile water with gentle stirring. Xylitol is added and mixed until completely dissolved. If using colloidal silver, the pre-prepared colloidal silver suspension is added. Grapefruit seed extract is added while mixing. If necessary, pH is adjusted using sodium bicarbonate or other suitable buffer. The solution is passed through a 0.22-micron sterile filter and introduced into sterile containers under aseptic conditions. The containers are sealed and labeled appropriately.

The composition(s) may be administered intranasally in an effective amount using any suitable delivery approach, including a metered-dose sprayer, atomizer, drops, rinse/irrigation, lavage, or nebulization. Dosing can be expressed as one or more actuations, sprays, or delivered volumes per nostril or per nasal cavity per administration, and may be repeated once or multiple times per day, or on another schedule selected based on severity, tolerability, and clinical response. In certain embodiments, the composition is delivered using a metered-dose device configured to deliver approximately 50 to 200 microliters per actuation, preferably 75 to 150 microliters.

In a two-step treatment system, the first and second compositions may be administered sequentially, concurrently, or separated by a time interval ranging from minutes to hours, depending on treatment protocol. For example, a two-step intranasal treatment can include a first step of administering a first intranasal composition comprising EDTA and xylitol formulated to disrupt biofilms; and a second step of administering a second intranasal composition comprising one or more antimicrobial agents selected from colloidal silver, grapefruit seed extract, and povidone iodine.

For MARCoNS treatment in CIRS patients, the composition(s) may be used multiple times daily for a period selected based on clinical response, with reassessment of nasal culture after treatment.

For maintenance and prevention of biofilm reformation, the composition(s) may be used on an ongoing basis according to patient need.

The concentrations disclosed herein are selected to balance antimicrobial efficacy with nasal tolerability. In preferred embodiments, EDTA concentrations above 1.0% are avoided to reduce mucosal irritation. Similarly, colloidal silver concentrations are maintained below 130 ppm to minimize epithelial toxicity while retaining antimicrobial effectiveness.

### WORKING EXAMPLE

A nasal spray formulation was prepared containing:
Disodium EDTA: 0.5%
Xylitol: 7.5%
Colloidal silver: 30 ppm
Grapefruit seed extract: 0.5%
Sterile water: to 100%

The efficacy of this formulation was evaluated using nasal swab samples from an individual previously positive for staphylococcal colonization. Two mannitol salt agar (MSA) plates were prepared:
Plate A (Control): Nasal swab sample plated directly without treatment
Plate B (Treated): Nasal swab sample treated with the BioFilm Clear formulation before plating

Both plates were incubated at room temperature for 7 days. Results showed:
Plate A: Multiple yellow colonies indicating staphylococcal growth
Plate B: No microbial growth observed

These results demonstrate the antimicrobial and anti-biofilm activity of the formulation against clinically relevant nasal pathogens. Although a single representative example is shown, similar reductions in microbial growth have been observed in additional evaluations.

The intranasal compositions of the present invention provide several advantages over existing treatments. For example, the combination of EDTA, xylitol, and antimicrobial agents attacks biofilms through multiple complementary mechanisms. The agents work synergistically, with EDTA disrupting the biofilm matrix to allow better penetration of antimicrobial agents. The formulation is effective against various biofilm-forming organisms including Staphylococcus species, Pseudomonas, and fungi. Unlike antibiotics, the antimicrobial agents used (colloidal silver, GSE) do not readily induce bacterial resistance. All components have established safety for nasal use, allowing for long-term treatment when necessary. Xylitol provides a cooling, moisturizing effect that improves tolerability.

What is claimed is:

1. A method for treating or reducing biofilm-associated nasal or sinonasal colonization in a subject, comprising:
(a) intranasally administering a first composition comprising ethylenediaminetetraacetic acid (EDTA) or a pharmaceutically acceptable salt thereof and xylitol, in an amount effective to weaken and/or disrupt a microbial biofilm; and
(b) intranasally administering a second composition comprising one or more antimicrobial agents, in an amount effective to reduce or eliminate one or more pathogens exposed by step (a).

2. The method of claim 1, wherein step (b) is performed after step (a) with a time interval in a range from minutes to hours.

3. The method of claim 1, wherein the one or more antimicrobial agents comprise colloidal silver, grapefruit seed extract, povidone iodine, essential oils, plant-derived phenolics, antimicrobial peptides, or combinations thereof.

4. The method of claim 1, wherein the first composition comprises about 0.05% to about 2.0% by weight EDTA and about 1% to about 20% by weight xylitol.

5. The method of claim 4, wherein the first composition comprises about 0.5% to about 1.0% by weight disodium EDTA and about 7.5% to about 10% by weight xylitol.

6. The method of claim 1, wherein the second composition comprises colloidal silver in an amount from about 1 ppm to about 300 ppm.

7. The method of claim 6, wherein the colloidal silver is present at about 20 ppm to about 130 ppm.

8. The method of claim 1, wherein the second composition comprises grapefruit seed extract in an amount from about 0.05% to about 2.0% by weight.

9. The method of claim 8, wherein the grapefruit seed extract is present at about 0.5% to about 1.0% by weight.

10. The method of claim 1, wherein intranasally administering comprises administration as a spray, mist, aerosol, drops, rinse, irrigation, lavage, or nebulization.

11. The method of claim 1, wherein administration comprises one or more actuations per nostril per administration.

12. The method of claim 1, wherein the biofilm-associated nasal or sinonasal colonization comprises Multiple Antibiotic Resistant Coagulase Negative Staphylococci (MARCoNS).

13. The method of claim 1, wherein the subject has Chronic Inflammatory Response Syndrome (CIRS).

14. A two-step intranasal treatment kit comprising:
(a) a first intranasal composition comprising EDTA or a pharmaceutically acceptable salt thereof and xylitol; and
(b) a second intranasal composition comprising one or more antimicrobial agents selected from colloidal silver, grapefruit seed extract, povidone iodine, and combinations thereof.

15. The kit of claim 14, wherein the first intranasal composition comprises about 0.05% to about 2.0% by weight EDTA and about 1% to about 20% by weight xylitol.

16. The kit of claim 14, wherein the second intranasal composition comprises colloidal silver in an amount from about 1 ppm to about 300 ppm and/or grapefruit seed extract in an amount from about 0.05% to about 2.0% by weight.

17. An intranasally administrable composition for disrupting biofilms and reducing pathogenic colonization, comprising:
(a) ethylenediaminetetraacetic acid (EDTA) or a pharmaceutically acceptable salt thereof;
(b) xylitol;
(c) at least one antimicrobial agent selected from the group consisting of colloidal silver, grapefruit seed extract, povidone iodine, and combinations thereof; and
(d) an aqueous carrier.

18. The composition of claim 17, wherein the EDTA salt is disodium EDTA.

19. The composition of claim 17, wherein the composition comprises: 0.5% to 2.0% by weight disodium EDTA; 1% to 20% by weight xylitol; colloidal silver at 1 ppm to 300 ppm; and
grapefruit seed extract at 0.5% to 2.0% by weight.

20. The composition of claim 17, wherein the composition comprises: 0.5% to 1.0% by weight disodium EDTA; 7.5% to 10% by weight xylitol; colloidal silver at 20 ppm to 130 ppm;
and grapefruit seed extract at 0.5% to 1.0% by weight.

21. The composition of claim 17, further comprising sodium chloride at a concentration sufficient to produce an isotonic solution.

22. The composition of claim 21, wherein the sodium chloride concentration is about 0.9% by weight.

23. The composition of claim 17, further comprising a buffering agent to maintain a pH between 6.0 and 7.0.

24. The composition of claim 23, wherein the buffering agent comprises sodium bicarbonate and the pH is maintained between 6.3 and 6.8.

25. The composition of claim 17, wherein the colloidal silver comprises silver nanoparticles having a size range of 1 to 30 nanometers.

26. The composition of claim 17, wherein the composition is preservative-free and packaged in a sterile, multi-dose container.
